(19) United States
(12) Patent Application Publication
(10) Pub. No.: US 2021/0196830 A1
(43) Pub. Date: Jul. 1, 2021

(54) GLYPISOME AS AN ENHANCER OF ANGIOGENIC GROWTH FACTOR ACTIVITY

(71) Applicant: Board of Regents, The University of Texas System, Austin, TX (US)

(72) Inventors: Aaron Baker, Austin, TX (US); Anthony Monteforte, Austin, TX (US)

(21) Appl. No.: 17/174,641

(22) Filed: Feb. 12, 2021

Related U.S. Application Data
(63) Continuation of application No. 14/723,012, filed on May 27, 2015, now abandoned.
(60) Provisional application No. 62/004,062, filed on May 28, 2014.

(51) Int. Cl. A61K 47/42; A61K 38/18; A61K 9/107; A61K 38/30; A61K 38/17; A61K 9/127; A61K 9/00; A61K 9/16; A61K 9/50

(52) U.S. Cl. CPC A61K 47/42; A61K 38/18; A61K 38/1858; A61K 38/1866; A61K 38/185; A61K 9/107; A61K 9/5036; A61K 38/1825; A61K 38/1709; A61K 9/1272; A61K 9/0019; A61K 9/1652; A61K 38/30

(57) ABSTRACT

Disclosed herein are proteovesicles, referred to herein as a “glypisomes”, that comprise a recombinant glypican polypeptide embedded in a lipid vesicle. Also disclosed is the use of these glypisomes to enhance the activity of growth factors.

### CROSS-REFERENCE TO RELATED APPLICATIONS

This application claims benefit of U.S. Provisional Application No. 62/004,062, filed May 28, 2014, the disclosure of which is hereby incorporated herein by reference in its entirety.

### STATEMENT REGARDING FEDERALLY SPONSORED RESEARCH OR DEVELOPMENT

This invention was made with Government Support under Grant No. OD008716 awarded by the National Institutes of Health. The Government has certain rights in the invention.

### BACKGROUND

Peripheral arterial disease (PAD) affects about 30 million people worldwide and it is estimated to affect over 16% of the general population over 65 years of age. Severe PAD has serious clinical consequences for patients including the formation of ulcers, pain from intermittent claudication and, ultimately, increased risk for limb amputation. The current clinical treatments for PAD include surgical revascularization with bypass grafting/endartectomy and percutaneous intervention such as angioplasty/stenting and catheter-based atherectomy. These treatments can provide temporary relief for patients with ischemia but ultimately remain limited in the long term by restenosis and further development of vascular disease. An alternative approach to the treatment of ischemic disease is to stimulate the body to create new vasculature to restore blood flow through its own regenerative processes. Several approaches have been explored to this end including the delivery of progenitor cells, viral vectors to express growth factor/angiogenic transcription factor genes, or through the delivery of growth factors. Growth factors as protein therapeutics for ischemia have the advantage of being appealing from a regulatory, production, and delivery viewpoint. However, in practice, angiogenic growth factor therapies, both through delivered proteins and genes, have met with disappointing success in treating patients. Thus, while the concept of therapeutic angiogenesis has great promise there are no current therapeutics that are capable of stimulating neovascularization in the context of human ischemic disease.

### SUMMARY

Disclosed herein are proteovesicles, referred to herein as “glypisomes” that comprise a recombinant glypican polypeptide embedded in a lipid vesicle or self-organized through a detergent extraction/removal process. For example, in some embodiments, the lipid vesicle is formed from detergent extraction of the recombinant glypican polypeptide from a cell. Therefore, in some embodiments, the lipid vesicle is a micelle or liposome.

Examples of glypicans include glypican-1, glypican-2, glypican-3, glypican-4, glypican-5, and glypican-6. The glypican in the glypisome can be selected based on the cell being targeted and the growth factor to be enhanced.

The disclosed proteovesicles can also be encapsulated along with a growth factor, such as a heparin-binding growth factor, into a biodegradable microcapsule or microbead for sustained co-release of the growth factor and proteovesicles in a subject. In some embodiments, the microcapsule or microbead comprises a biocompatible hydrogel, such as a polysaccharide hydrogel. For example, the microcapsule or microbead can comprise alginate gel, collagen gel, fibrin gel, poly(lactic-co-glycolic acid) (PLGA), or any mixture thereof.

The microcapsules or microbeads can be any size suitable to encapsulate the proteovesicle proteovesicles and growth factors. For example, the microcapsules or microbeads can be from 1 μm in diameter, up to 3 mm in diameter, including about 1 μm to 100 μm, 100 μm to 1 mm, or 1 mm to 3 mm.

The amount of proteovesicles and growth factors in the microcapsules or microbeads can be individually selected based upon the specific growth factors being used, release rates of the biodegradable microcapsules or microbeads, and requirements of the target tissue.

Each proteovesicle can comprise from about 100 ng/ml up to about 100 μg/ml lipid, including about 100 ng/ml, 1 μg/ml, 10 μg/ml, 100 μg/ml, or any amount in-between. Each proteovesicle can comprise from about 5 ng/ml up to about 5 μg/ml glypican, including about 5 ng/ml, 50 μg/ml, 500 ng/ml, 5 μg/ml, or any amount in-between. In some embodiments, the ratio of lipids to glypican is preferably maintained as the amount of glypican is adjusted.

In some embodiments, the growth factor is a heparin-binding growth factor. For example, the growth factor can be an angiogenesis-related or wound healing-related growth factor. Non-limiting examples of angiogenesis-related growth factors include fibroblast growth factors (FGFs), platelet-derived growth factors (PDGFs), vascular endothelial growth factors (VEGFs), and Placental growth factors (PlGFs).

In some embodiments, the growth factor is a neurotrophic growth factor. Non-limiting examples of neurotrophic growth factor include nerve growth factors (NGFs), FGFs, brain-derived neurotrophic factors (BDNFs), insulin-like growth factors (IGFs), ciliary neurotrophic factors (CNTFs), and neurotrophic factor-4/5 (NT-4/5).

Other factors include those of the bone morphogenetic proteins (BMPs), transforming growth factors (TGFs), tumor necrosis factors (TNF), interleukins (ILs), monocyte chemotactic proteins (MCPs), insulin, insulin-like growth factors (IGFs), WNT, notch, Epidermal growth factors (EGFs), EGF-like growth factor (HB-EGF), slit proteins, semaphorins, cytokines, and chemokines.

Also disclosed is the use of these glypisomes to enhance the angiogenic, neurotrophic, or other such activity of growth factors. For example, disclosed is a method for enhancing the angiogenic activity of a growth factor in a subject, comprising administering to a subject in need thereof a proteovesicle disclosed herein. Also disclosed is a method for promoting angiogenesis in a subject, comprising administering to a subject in need thereof therapeutically effective amount of a disclosed microcapsule or microbead encapsulating a proteovesicle and an angiogenesis-related growth factor. For example, the method can be used to treat a subject that has been diagnosed with peripheral arterial disease (PAD), chronic wounds, or an ischemic cardiovascular or cerebrovascular disorder. Also disclosed is a method for promoting nerve regeneration in a subject, comprising administering to a subject in need thereof therapeutically effective amount of a disclosed microcapsule or microbead encapsulating a proteovesicle and a neurotrophic growth factor.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIGS. 1A and 1B are bar graphs showing proliferation of human endothelial cells after stimulation with growth factors and glypisomes with varying ratios between lipid and recombinant glypcian-1 (GPC-1). Endothelial cells were treated with 10 ng/ml FGF-2 (FIG. 1A) or 10 ng/ml VEGF165 (FIG. 1B) and glypisomes, and then proliferation was measured from Brdu incorposation assay. *Statistically significant difference between group and the no growth factor group (p<0.05). †Statistically significant different from the no growth factor and growth factor alone groups (p<0.05).

FIGS. 2A to 2D are bar graphs (FIGS. 2A to 2C) and microscopy images (FIG. 2D) showing results of a tube formation assay performed by seeding human endothelial cells onto growth factor reduced matrigel and treating the cells with glypisomes with varying composition and 10 ng/ml FGF-2. The formation of tubes was assessed by phase contrast microscopy. FIGS. 2A to 2C show the number of branching points (FIG. 2A), tube length (FIG. 2B), and tube number (FIG. 2C) for varying concentrations of lipid and glypican-1 (GPC-1). *Statistically significant difference between group and the no growth factor group (p<0.05). †Statistically significant different from the no growth factor and growth factor alone groups (p<0.05). Scale Bar in FIG. 2D=200 μm.

FIG. 3A to 3D are bar graphs and microscopy images showing results of an in-vitro angiogenesis assay performed by seeding human endothelial cells onto growth factor reduced matrigel and treating the cells with glypisomes with varying composition and 10 ng/ml VEGF165. The formation of tubes was assessed by phase contrast microscopy. FIGS. 3A to 3C show the number of branching points (FIG. 3A), tube length (FIG. 3B), and tube number (FIG. 3C) for varying concentrations of lipid and glypican-1 (GPC-1). *Statistically significant difference between group and the no growth factor group (p<0.05). †Statistically significant different from the no growth factor and growth factor alone groups (p<0.05). Scale Bar in FIG. 3D=200 μm.

FIGS. 4A to 4D are bar graphs and microscopy images showing results of an in vitro wound healing assay using glypisomes and growth factors. Human endothelial cells were grown to post-confluence and then a scratch wound was created using a cell scraper. The migration/closure of the wound was measured over time after wounding. Shown in FIGS. 4A and 4B are the total distance (μm) migrated by the wound edges. Cells were treated with FGF-2 (FIG. 4A, 4C) or VEGF (FIG. 4B, 4D) at the time of injury. The glypisomes (G1PL) used were the optimal composition glypisomes determined from activity in the proliferation/tube formation assays. *Statistically significant different from the no growth factor and growth factor alone groups (p<0.05). Scale Bar in FIGS. 4C, 4D=200 μm.

FIG. 5A is an image of glypisomes encapsulated in alginate beads. FIG. 5B is an image of alginate/glypisomes implanted in the hind limb of mice after Ischemia was induced by femoral artery ligation. FIG. 5C is a series of laser speckle contrast images used to assess blood perfusion in the feet of the mice over time that were given either alginate beads with just FGF-2 (left) or FGF-2 and glypisomes (G1PL) (right) on day 1 (top) and day 14 (bottom). FIG. 5D is a graph showing quantitative analysis of the perfusion of the feet after induction of hind limb ischemia and treatment with FGF-2 (solid line, squares) or FGF-2 and G1PL (dashed line, circles). Relative blood flow was defined as the speckle contrast ratio between the ischemic limb and the control limb. *Statistically significant difference between group and FGF-2 alone group at the same time point (p<0.05).

FIGS. 6A and 6B are images of from histological analysis of the calf and thigh muscle of the ischemic limb after 14 days of treatment with FGF-2 or FGF-2 and glyposomes (G1PLs). FIG. 6A shows that ischemic changes including the loss of muscle of fibers was dramatically reduced in the calf muscle with FGF-2 and glypisome treatment in comparison to FGF-2 alone. Ischemic changes included the loss of muscle fibers/altered morphology with the tissue. There were fewer regions with lost muscle fibers in the thigh than the calf in FGF-2 treated mice. A quantitative analysis of the fibers that had ischemic changes revealed markedly reduced incidence of ischemic changes in the fibers in the FGF-2 with glypisomes group (FIG. 6C). In addition, there was an increased number of blood vessels in the ischemic calf and thigh from these animals (FIGS. 6D and 6E). Scale Bar in FIGS. 6A, 6B=100 μm.

FIG. 7 is a silver stained gel of isolated of recombinant glypican-1. Lane 1: Whole lysates from glypican-1 overexpressing cells. Lane 2: Isolated glypican-1.

FIG. 8A is a diagram of glypican-1 anchored to a cell membrane by a glycosylphosphatidylinositol (GPI anchor) and interacting with glycosaminoglycan (GAG). FIG. 8B is a diagram of glypisome-1 embedded into a liposome, referred to as a glypisome.

FIG. 9 shows dynamic light scattering (DLS) on glypisomes with varying lipid to glypican ratios. For example the notation L40:G60 denotes a mixture of 40% liposomes (12.3 mM lipid) with 60% glypican-1 solution (61 μg/ml). Note that L0:G100 is the isolated recombinant glypican-1 protein and that this has a hydrodynamic radius much larger than a glypican-1 monomer, implying self-association of the protein.

### DETAILED DESCRIPTION

The physiological processes of angiogenesis, vasculogenesis, and arteriogenesis contribute to the growth of collateral vessels in response to obstructive arterial disease causing lower limb or myocardial ischaemia. However, in clinical practice, the endogenous angiogenic response is often suboptimal or impaired, e.g. by factors such as ageing, diabetes or drug therapies. Therapeutic angiogenesis is an application of biotechnology to stimulate new vessel formation via local administration of pro-angiogenic growth factors in the form of recombinant protein or gene therapy, or by implantation of endothelial progenitor cells that will synthesize multiple angiogenic cytokines. Numerous experimental and clinical studies have sought to establish ‘proof of concept’ for therapeutic angiogenesis in PAD and myocardial ischaemia using different treatment modalities, but the results have been inconsistent.

One potential reason for angiogenic therapies to fail in clinical trials is the presence of disease-induced changes in the signaling and functional response of tissues to angiogenic stimuli. In this view, disease processes that produce ischemia and common co-morbities, such as diabetes and hyperlipidemia, also induce disruptions in the pathways that are critical to angiogenesis. Insulin resistance is a hallmark of diabetic disease and in the same way ischemia in aged humans may also represent a state in which the body can no longer respond effectively to growth factors such as FGF-2 and VEGF. Many of the heparan sulfate proteoglycans that are co-receptors of the FGF and VEGF families of growth factors are expressed at lower levels in Ob/Ob mice than WT mice where both on a high fat diet that induces vascular disease. In addition, heparanase expression is increased in cells treated with fatty acid and animals on a high fat diet, in atherosclerotic plaques, and following stenting or vascular injury.

Disclosed herein are compositions and methods to compensate for disease-induced loss of cell surface heparan sulfate proteoglycans (HSPGs). The HSPGs are complex molecules that consist of a core protein with one or more heparan sulfate glycosaminoglycan chains attached. The binding and activity of many growth factors is altered by the presence of cell surface or extracellular matrix heparan sulfate proteoglycans. In many cases, heparan sulfate binding serves to stabilize receptor interactions and with the HSPG acting as a co-receptor.

As disclosed herein, the cell-surface proteoglycan glypican can be used as a therapeutic enhancer for growth factors. The glypicans are distinguished from other cell surface HSPGs such as the syndecans by their linkage to the membrane through a glycosylphosphatidylinositol (GPI) anchor. This GPI linker enables the phospholipase mediated-shedding of glypicans and drives preferential localization of the protein within cholesterol-rich lipid rafts. These properties allow glypicans to associate with calveolae, control endocytosis/recycling and transcellular transport, regulate the formation of morphogen gradients, and cell signaling. Glypican-1 is highly expressed in glioma cells and their associated vasculature. A hallmark of gliomas is vigorous angiogenic response that drives tumor neovascularization through multiple mechanisms. Glypican-1 is the prevalent member of the glypican family in endothelial cells and the vascular system. Glypican-1 expression has been found to play a role in the growth, metastasis and angiogenic properties of gliomas. Glypican-1 can act as a co-receptor/modulator for many angiogenic factors including members of the FGF and VEGF growth factor. In addition, glypican-1 can stimulate cell cycle progression in endothelial cells by regulating cell cycle progression.

Glypisomes

Therefore, disclosed is a proteovesicle, referred to herein as a “glypisome” that comprises a recombinant glypican polypeptide embedded in a lipid vesicle. Also disclosed is the use of these glypisomes to enhance the angiogenic, neurotrophic, or other such activity of growth factors.

Heparin-Dependant Growth Factors

Numerous inducers of angiogenesis have been identified, including the members of the vascular endothelial growth factor (VEGF) family. Different isoforms of mammalian VEGFs interact with tyrosine kinase VEGF receptors (VEGFRs) expressed on the surface of endothelial cells (ECs) and with heparan sulfate (HS) proteoglycan (HSPG) and neuropilin (NRP) coreceptors, thus activating a proangiogenic response.

HSPGs modulate the interaction of proangiogenic growth factors with their receptors, such as VEGFs binding to VEGF receptor-2 (VEGFR2) and neuropilin coreceptors in endothelial cells (ECs). HSPGs consist of a core protein and of glycosaminoglycan (GAG) chains represented by un-branched heparin-like polysaccharides. They are found in free forms, in the extracellular matrix (ECM), or associated with the plasma membrane where they regulate the function of a wide range of ligands. In particular, endothelial HSPGs modulate angiogenesis by affecting bioavailability and interaction of VEGFs with signaling VEGFRs and NRP coreceptors. Heparin/HS interaction with angiogenic growth factors depends on the degree/distribution of sulfate groups and length of the GAG chain, distinct oligosaccharide sequences mediating its binding activity.

Examples of angiogenesis-related growth factors, cytokines, and chemokines include: Fibroblast growth factors (FGFs), Platelet-derived growth factor (PDGF), Vascular endothelial growth factor (VEGF), Pleiotrophin, Placental growth factor (PlGF), Platelet factor-4 (PF-4), EGF-like growth factor, Interleukin-8 (IL-8), Hepatocyte growth factor (HGF), Macrophage inflammatory protein-1 (MIP-1), Transforming growth factor-beta (TGF-beta), Interferon-g-inducible protein-10 (IP-10), Interferon-gamma (IFN-gamma), and HIV-Tat transactivating factor.

Angiogenic growth factors induce response in target endothelial cells by binding to cognate cell-surface tyrosine kinase (TK) receptors. The interaction of growth factors to TK receptors is modulated by HSPGs. For instance, the interaction of FGF-2 or of the VEGF165 isoform to TK receptors is strongly reduced in cells made HSPG-deficient by treatment with heparinase or chlorate.

In some embodiments, the growth factor is a neurotrophic growth factor. Non-limiting examples of neurotrophic growth factor include nerve growth factors (NGFs), FGFs, brain-derived neurotrophic factors (BDNFs), insulin-like growth factors (IGFs), ciliary neurotrophic factors (CNTFs), and neurotrophic factor-4/5 (NT-4/5).

Other factors include those of the bone morphogenetic proteins (BMPs), transforming growth factors (TGFs), tumor necrosis factors (TNF), interleukins (ILs), monocyte chemotactic proteins (MCPs), insulin, insulin-like growth factors (IGFs), WNT, notch, Epidermal growth factors (EGFs), EGF-like growth factor (HB-EGF), slit proteins, semaphorins, cytokines, and chemokines.

The disclosed glypisomes can be used to enhance the activity of any one or more of these growth factors.

Glypicans

Glypicans constitute one of the two major families of heparan sulfate proteoglycans, with the other major family being syndecans. Six glypicans have been identified in mammals, and are referred to as glypican-1 to glypican-6 (GPC1 to GPC6). While six glypicans have been identified in mammals, several characteristics remain consistent between these different proteins. First, the core protein of all glypicans is similar in size, approximately ranging between 60 and 70 kDa. Additionally, in terms of amino acid sequence, the location of fourteen cysteine residues is conserved. It is thought that the fourteen conserved cysteine residues play a vital role in determining three-dimensional shape, thus suggesting the existence of a highly similar three-dimensional structure. Overall, GPC3 and GPC5 have very similar primary structures with 43% sequence similarity. On the other hand, GPC1, GPC2, GPC4, and GPC6 have between 35% and 63% sequence similarity. Thus, GPC3 and GPC5 are often referred to as one subfamily of glypicans, with GPC1, GPC2, GPC4, and GPC6 constituting the other group. The amino acid sequence and structure of each glypican is well-conserved between species; it has been reported that all vertebrate glypicans are more than 90% similar regardless of the species.

Glypican-1 is encoded by the GPC1 gene. Human glypican-1 can have the amino acid sequence found in Accession No. NP_002072. Glypican-2 is encoded by the GPC2 gene. Human glypican-1 can have the amino acid sequence found in Accession No. NP_689955. Glypican-3 is encoded by the GPC3 gene. Human glypican-1 can have the amino acid sequence found in Accession No. NP_001158089. Glypican-4 is encoded by the GPC4 gene. Human glypican-1 can have the amino acid sequence found in Accession No. NP_001439. Glypican-5 is encoded by the GPC5 gene. Human glypican-1 can have the amino acid sequence found in Accession No. NP_004457. Glypican-6 is encoded by the GPC6 gene. Human glypican-1 can have the amino acid sequence found in Accession No. NP_005699.

Also disclosed are peptide variants and/or fragments of naturally occurring glypicans. For example, the disclosed glypisomes can include peptides having amino acid sequences that are at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical to a naturally occurring sequence, such as the reference sequences disclosed herein.

For all members of the glypican family, the C-terminus of the protein is incorporated into the cell membrane through a glycosylphosphatidylinositol (GPI) anchor that is added as a post-translational modification to the protein. This is in contrast to other cell surface heparan sulfate proteoglycans such as the syndecan family proteins. To facilitate for the addition of the GPI anchor, glypicans have a hydrophobic domain at the C-terminus of the protein. Within 50 amino acids of this GPI anchor, the heparan sulfate chains attach to the protein core. Therefore, unlike syndecans, the GAG chains attached to glypicans are located rather close to the cell-membrane. The glypicans found in vertebrates, Drosophila, and C. elegans all have an N-terminal signal sequence.

Glycosylphosphatidylinositol (GPI anchor) is a glycolipid that can be attached to the C-terminus of a protein during posttranslational modification. It is composed of a phosphatidylinositol group linked through a carbohydrate-containing linker (glucosamine and mannose glycosidically bound to the inositol residue) and via an ethanolamine phosphate (EtNP) bridge to the C-terminal amino acid of a mature protein. The two fatty acids within the hydrophobic phosphatidyl-inositol group anchor the protein to the cell membrane.

Glypiated (GPI-linked) proteins (e.g., glypicans) contain a signal peptide, thus directing them into the endoplasmic reticulum (ER). The C-terminus is composed of hydrophobic amino acids that stay inserted in the ER membrane. The hydrophobic end is then cleaved off and replaced by the GPI-anchor. As the protein processes through the secretory pathway, it is transferred via vesicles to the Golgi apparatus and finally to the extracellular space where it remains attached to the exterior leaflet of the cell membrane. Since the glypiation, the addition of the GPI tail, is the sole means of attachment of such proteins to the membrane, cleavage of the group by phospholipases will result in controlled release of the protein from the membrane.

Therefore, also disclosed are any peptide fragments of the naturally occurring glypicans or variants thereof, as discussed above, that 1) can maintain the ability to carry heparan sulfate glycosaminoglycan (GAG) chains, and 2) can be GPI-linked into a lipid vesicle membrane.

The glypican can be extracted from natural sources or produced synthetically. However, in some embodiments, the glypican is produced recombinantly by incorporating a nucleic acid encoding a GPC gene into an expression vector, such that it is operably linked to an expression control sequence. A suitable cell line transformed with this expression vector can be cultured to produce large amounts of the glypican protein, which can be isolated by, for example, the methods described below.

Lipid Vesicle

The disclosed glypisome comprises glypican embedded in the membrane of a lipid vesicle, which can then enhance the activity of an angiogenesis-related growth factor. All that is required for a polypeptide to be considered embedded within a lipid vesicle is that a portion of the polypeptide, for example, hydrophobic residues of the polypeptide, be in contact with the hydrophobic moieties such that the polypeptide is stably associated with the lipid vesicle. In some embodiments, the lipid vesicle may be a micelle or liposome in which a glypican polypeptide is embedded by means of the hydrophobic interactions between the GPI anchor of the glypican and the lipid portion of the liposome or micelle.

A lipid vesicle may comprise phospholipids, glycolipids, steroids, or synthetic lipid analogues (e.g., amphipathic, synthethic polymers, such as poly(2-methyl-2-oxazoline) (PMOZ) and poly(2-ethyl-2-oxazoline) (PEOZ)). A lipid vesicle that comprises phospholipids may exist as a monolayer or a bilayer. Modifications may be made to a lipid-based vehicle to increase the efficiency with which the lipid vesicle fuses with a cell, for example, by changing the lipid content. A lipid vesicle may be a micelle or a bacterial or red cell ghost. A lipid vesicle may be vesicles or membrane fragments of transgenic cells. The lipid vesicle may be a liposome, which is a general category of vesicle that may comprise one or more lipid bilayers surrounding an aqueous space. Liposomes include unilamellar vesicles composed of a single membrane or a lipid bilayer, and multilamellar vesicles (MLVs) composed of many concentric membranes (or lipid bilayers).

Since recombinant glypicans are embedded into the cell membranes of host cells, the protein must be extracted by detergents in order to be isolated it from the cells. Generally, moderate concentrations of mild (i.e., nonionic) detergents compromise the integrity of cell membranes, thereby facilitating lysis of cells and extraction of soluble protein, often in native form. Using certain buffer conditions, various detergents effectively penetrate between the membrane bilayers at concentrations sufficient to form mixed micelles with isolated phospholipids and membrane proteins.

Denaturing detergents such as SDS bind to both membrane (hydrophobic) and nonmembrane (water-soluble, hydrophilic) proteins at concentrations below the CMC, i.e. as monomers. The reaction is equilibrium-driven until saturated. Therefore, the free concentration of monomers determines the detergent concentration. SDS binding is cooperative (the binding of one molecule of SDS increases the probability that another molecule of SDS will bind to that protein) and alters most proteins into rigid rods whose length is proportional to molecular weight.

Non-denaturing detergents such as Triton X-100 have rigid and bulky nonpolar heads that do not penetrate into water-soluble proteins; consequently, they generally do not disrupt native interactions and structures of water-soluble proteins and do not have cooperative binding properties. The main effect of non-denaturing detergents is to associate with hydrophobic parts of membrane proteins, thereby conferring miscibility to them.

At concentrations below the CMC, detergent monomers bind to water-soluble proteins. Above the CMC, binding of detergent to proteins competes with the self-association of detergent molecules into micelles. Consequently, there is effectively no increase in protein-bound detergent monomers with increasing detergent concentration beyond the CMC.

Detergent monomers solubilize membrane proteins by partitioning into the membrane bilayer. With increasing amounts of detergents, membranes undergo various stages of solubilization. The initial stage is lysis or rupture of the membrane. At detergent:membrane lipid molar ratios of 0.1:1 through 1:1 the lipid bilayer usually remains intact but selective extraction of some membrane proteins occurs. Increasing the ratio to 2:1, solubilization of the membrane occurs resulting in mixed micelles. These include phospholipid-detergent micelles, detergent-protein micelles, and lipid-detergent-protein micelles. At a ratio of 10:1, all native membrane lipid:protein interactions are effectively exchanged for detergent:protein interactions. The amount of detergent needed for complete protein extraction depends on the CMC, aggregation number, temperature and nature of the membrane and the detergent.

In some embodiments, lipid vesicle of the glypisome is formed from detergent extraction of the recombinant glypican polypeptide from a cell. For example, detergent extraction can be used to lyse the cells expressing the glypican such that the proteins are extracted into vesicles, such as micelles. These micelles can then be used as lipid vesicles to deliver the glypicans to cells containing angiogenesis-related growth factors.

In other embodiments, the glypican polypeptides are fully extracted from the lipids of the cells, which involves removing the detergent from the solubilized proteins. Detergent removal can be attempted in a number ways. Dialysis is effective for removal of detergents that have very high CMCs and/or small aggregation numbers, such the N-octyl glucosides. Detergents with low CMCs and large aggregation numbers cannot be dialyzed since most of the detergent molecules will be in micelles that are too large to diffuse through the pores of the dialysis membrane; only excess monomer can be dialyzed. Ion exchange chromatography using appropriate conditions to selectively bind and elute the proteins of interest is another effective way to remove detergent. Sucrose density gradient separation also can be used. Once extracted from the cells and the detergent, glypican can be embedded in a lipid vesicle, such as a liposome, using routine methods.

Microcapsules or Microbeads

The disclosed glypisomes can be administered to a subject alone or in combination with one or more angiogenesis-related growth factors. Therefore, also disclosed is a composition comprising a glypisome and one or more growth factors. In some embodiments, the glypisome and one or more growth factors are encapsulated together in a microcapsule or microbead. For example, in some embodiments, the microcapsule or microbead comprises a biocompatible hydrogel.

Compositions that form hydrogels generally fall into three classes. The first class carries a net negative charge and is typified by alginate. The second class carries a net positive charge and is typified by extracellular matrix components, such as collagen and laminin. Examples of commercially available extracellular matrix components include Matrigel™ and Vitrogen™. The third class is net neutral in charge. An example of a net neutral hydrogel is highly crosslinked polyethylene oxide, or polyvinyalcohol.

Examples of materials which can be used to form a suitable hydrogel include polysaccharides such as alginate, polyphosphazines, poly(acrylic acids), poly(methacrylic acids), poly(alkylene oxides), poly(vinyl acetate), poly(acrylamides) such as poly(N-isopropylacrylamide), polyvinylpyrrolidone (PVP), and copolymers and blends of each. In some embodiments, block copolymers can be used. For example, poloxamers containing a hydrophobic poly(alkylene oxide) segment (i.e., polypropylene oxide) and hydrophilic poly(alkylene oxide) segment (i.e., polyethylene oxide) can be used. Polymers of this type are available are known in the art, and commercially available under the trade name PLURONICS from BASF. In some embodiments, the material is selected such that it forms a thermally responsive hydrogel.

In general, the polymers are at least partially soluble in aqueous solutions, such as water, buffered salt solutions, or aqueous alcohol solutions. In some embodiments, the polymers have polar groups, charged groups, acidic groups or salts thereof, basic groups or salts thereof, or combinations thereof. Examples of polymers with acidic groups poly(phosphazenes), poly(acrylic acids), poly(methacrylic acids), poly(vinyl acetate), and sulfonated polymers, such as sulfonated polystyrene. Copolymers having acidic side groups formed by reaction of acrylic or methacrylic acid and vinyl ether monomers or polymers can also be used. Examples of acidic groups include carboxylic acid groups and sulfonic acid groups.

Examples of polymers with basic groups include poly(vinyl amines), poly(vinyl pyridine), poly(vinyl imidazole), and some imino substituted polyphosphazenes. Nitrogen-containing groups in these polymers can be converted to ammonium or quaternary salts. Ammonium or quaternary salts can also be formed from the backbone nitrogens or pendant imino groups. Examples of basic groups include amino and imino groups.

In certain embodiments, the biocompatible hydrogel-forming polymer is a water-soluble gelling agent. In certain embodiments, the water-soluble gelling agent is a polysaccharide gum, such as a polyanionic polysaccharide. In some cases, glypisome and one or more growth factors are encapsulated using an anionic polymer such as alginate to form a microcapsule.

Mammalian and non-mammalian polysaccharides have been explored for cell encapsulation. These materials can be used, alone or in part, to form the microcapsule. Exemplary polysaccharides include alginate, chitosan, hyaluronan (HA), and chondroitin sulfate. Alginate and chitosan form crosslinked hydrogels under certain solution conditions, while HA and chondroitin sulfate are preferably modified to contain crosslinkable groups to form a hydrogel.

In some embodiments, the microcapsule or microbead comprises alginate or derivative thereof. Alginates are a family of unbranched anionic polysaccharides derived primarily from brown algae which occur extracellularly and intracellularly at approximately 20% to 40% of the dry weight. The 1,4-linked α-1-guluronate (G) and β-D-mannuronate (M) are arranged in homopolymeric (GGG blocks and MMM blocks) or heteropolymeric block structures (MGM blocks). Cell walls of brown algae also contain 5% to 20% of fucoidan, a branched polysaccharide sulphate ester with 1-fucose four-sulfate blocks as the major component. Commercial alginates are often extracted from algae washed ashore, and their properties depend on the harvesting and extraction processes. Although the properties of the hydrogel can be controlled to some degree through changes in the alginate precursor (molecular weight, composition, and macromer concentration), alginate does not degrade, but rather dissolves when the divalent cations are replaced by monovalent ions. In addition, alginate does not promote cell interactions.

Alginate can form a gel in the presence of divalent cations via ionic crosslinking. Crosslinking can be performed by addition of a divalent metal cation (e.g., a calcium ion or a barium ion), or by cross-linking with a polycationic polymer (e.g., an amino acid polymer such as polylysine). See e.g., U.S. Pat. Nos. 4,806,355, 4,689,293 and 4,673,566 to Goosen et al.; U.S. Pat. Nos. 4,409,331, 4,407,957, 4,391,909 and 4,352,883 to Lim et al.; U.S. Pat. Nos. 4,749,620 and 4,744,933 to Rha et al.; and U.S. Pat. No. 5,427,935 to Wang et al. Amino acid polymers that may be used to crosslink hydrogel forming polymers such as alginate include the cationic poly(amino acids) such as polylysine, polyarginine, polyornithine, and copolymers and blends thereof.

In some embodiments, the microcapsule or microbead comprises chitosan or derivative thereof. Chitosan is made by partially deacetylating chitin, a natural non-mammalian polysaccharide, which exhibits a close resemblance to mammalian polysaccharides, making it attractive for cell encapsulation. Chitosan degrades predominantly by lysozyme through hydrolysis of the acetylated residues. Higher degrees of deacetylation lead to slower degradation times, but better cell adhesion due to increased hydrophobicity. Under dilute acid conditions (pH<6), chitosan is positively charged and water soluble, while at physiological pH, chitosan is neutral and hydrophobic, leading to the formation of a solid physically crosslinked hydrogel. The addition of polyol salts enables encapsulation of cells at neutral pH, where gelation becomes temperature dependent. Chitosan has many amine and hydroxyl groups that can be modified. For example, chitosan has been modified by grafting methacrylic acid to create a crosslinkable macromer while also grafting lactic acid to enhance its water solubility at physiological pH. This crosslinked chitosan hydrogel degrades in the presence of lysozyme and chondrocytes. Photopolymerizable chitosan macromer can be synthesized by modifying chitosan with photoreactive azidobenzoic acid groups. Upon exposure to UV in the absence of any initiator, reactive nitrene groups are formed that react with each other or other amine groups on the chitosan to form an azo crosslink.

In some embodiments, the microcapsule or microbead comprises hyaluronan or derivative thereof. Hyaluronan (HA) is a glycosaminoglycan present in many tissues throughout the body that plays an important role in embryonic development, wound healing, and angiogenesis. In addition, HA interacts with cells through cell-surface receptors to influence intracellular signaling pathways. Together, these qualities make HA attractive for tissue engineering scaffolds. HA can be modified with crosslinkable moieties, such as methacrylates and thiols, for cell encapsulation. Crosslinked HA gels remain susceptible to degradation by hyaluronidase, which breaks HA into oligosaccharide fragments of varying molecular weights. Auricular chondrocytes can be encapsulated in photopolymerized HA hydrogels where the gel structure is controlled by the macromer concentration and macromer molecular weight. In addition, photopolymerized HA and dextran hydrogels maintain long-term culture of undifferentiated human embryonic stem cells. HA hydrogels have also been fabricated through Michael-type addition reaction mechanisms where either acrylated HA is reacted with PEG-tetrathiol, or thiol-modified HA is reacted with PEG diacrylate.

Chondroitin sulfate makes up a large percentage of structural proteoglycans found in many tissues, including skin, cartilage, tendons, and heart valves, making it an attractive biopolymer for a range of tissue engineering applications. Photocrosslinked chondroitin sulfate hydrogels can be been prepared by modifying chondroitin sulfate with methacrylate groups. The hydrogel properties were readily controlled by the degree of methacrylate substitution and macromer concentration in solution prior to polymerization. Further, the negatively charged polymer creates increased swelling pressures allowing the gel to imbibe more water without sacrificing its mechanical properties. Copolymer hydro gels of chondroitin sulfate and an inert polymer, such as PEG or PVA, may also be used.

In some embodiments, the microcapsule or microbead comprises a hydrogel that mimics an extracellular matrix (ECM). Components of an extracellular matrix can include for example collagen, fibrin, fibrinogen, thrombin, elastin, laminin, fibronectin, hyaluronic acid, chondroitin 4-sulfate, chondroitin 6-sulfate, dermatan sulfate, heparin sulfate, heparin, and keratan sulfate, and proteoglycans.

In some embodiments, the microcapsule or microbead comprises a synthetic polymer or polymers. Polyethylene glycol (PEG) has been the most widely used synthetic polymer to create macromers for cell encapsulation. A number of studies have used poly(ethylene glycol)di(meth)acrylate to encapsulate a variety of cells. Biodegradable PEG hydrogels can be been prepared from triblock copolymers of poly(α-hydroxy esters)-b-poly(ethylene glycol)-b-poly(α-hydroxy esters) endcapped with (meth)acrylate functional groups to enable crosslinking. PLA and poly(8-caprolactone) (PCL) have been the most commonly used poly(α-hydroxy esters) in creating biodegradable PEG macromers for cell encapsulation. The degradation profile and rate are controlled through the length of the degradable block and the chemistry. The ester bonds may also degrade by esterases present in serum, which accelerates degradation. Biodegradable PEG hydrogels can also be fabricated from precursors of PEG-bis-[2-acryloyloxy propanoate]. As an alternative to linear PEG macromers, PEG-based dendrimers of poly(glycerol-succinic acid)-PEG, which contain multiple reactive vinyl groups per PEG molecule, can be used. An attractive feature of these materials is the ability to control the degree of branching, which consequently affects the overall structural properties of the hydrogel and its degradation. Degradation will occur through the ester linkages present in the dendrimer backbone.

In some cases, the hydrogel-forming material is selected from the group consisting of poly-lactic-co-glycolic acid (PLGA), poly-l-lactide (PLLA), poly-caprolactone (PCL), polyglycolide (PGA), derivatives thereof, copolymers thereof, and mixtures thereof.

The biocompatible, hydrogel-forming polymer can contain polyphosphoesters or polyphosphates where the phosphoester linkage is susceptible to hydrolytic degradation resulting in the release of phosphate. For example, a phosphoester can be incorporated into the backbone of a crosslinkable PEG macromer, poly(ethylene glycol)-di-[ethylphosphatidyl (ethylene glycol) methacrylate] (PhosPEG-dMA), to form a biodegradable hydrogel. The addition of alkaline phosphatase, an ECM component synthesized by bone cells, enhances degradation. The degradation product, phosphoric acid, reacts with calcium ions in the medium to produce insoluble calcium phosphate inducing autocalcification within the hydrogel. Poly(6-aminoethyl propylene phosphate), a polyphosphoester, can be modified with methacrylates to create multivinyl macromers where the degradation rate was controlled by the degree of derivitization of the polyphosphoester polymer.

Polyphosphazenes are polymers with backbones consisting of nitrogen and phosphorous separated by alternating single and double bonds. Each phosphorous atom is covalently bonded to two side chains. The polyphosphazenes suitable for cross-linking have a majority of side chain groups which are acidic and capable of forming salt bridges with di- or trivalent cations. Examples of preferred acidic side groups are carboxylic acid groups and sulfonic acid groups. Hydrolytically stable polyphosphazenes are formed of monomers having carboxylic acid side groups that are crosslinked by divalent or trivalent cations such as Ca′ or Al′. Polymers can be synthesized that degrade by hydrolysis by incorporating monomers having imidazole, amino acid ester, or glycerol side groups. Bioerodible polyphosphazines have at least two differing types of side chains, acidic side groups capable of forming salt bridges with multivalent cations, and side groups that hydrolyze under in vivo conditions, e.g., imidazole groups, amino acid esters, glycerol and glucosyl. Hydrolysis of the side chain results in erosion of the polymer. Examples of hydrolyzing side chains are unsubstituted and substituted imidizoles and amino acid esters in which the group is bonded to the phosphorous atom through an amino linkage (polyphosphazene polymers in which both R groups are attached in this manner are known as polyaminophosphazenes). For polyimidazolephosphazenes, some of the “R” groups on the polyphosphazene backbone are imidazole rings, attached to phosphorous in the backbone through a ring nitrogen atom.

Therapeutic Angiogenesis

Insufficient angiogenesis is a hallmark feature of chronic wounds. Angiogenesis is often impaired in the elderly, in people with high cholesterol, diabetes, and in heavy drinkers and smokers. Certain medications can also impair angiogenesis, including some common pain medications, diuretics, and high blood pressure drugs. Age, high cholesterol, alcohol use, and diabetes are risk factors known to inhibit angiogenesis. Non-limiting examples of prescription medicines that are known to inhibit angiogenesis include: antibiotics (clarithromycin, doxycycline, tetracycline), high blood pressure medications (captopril, enalapril, metoprolol), diuretics (bumetanide, furosemide), nonsteroidal anti-inflammatory drugs (aspirin, ibuprofen), COX-2 inhibitors (celecoxib), and PPAR-+ agonists (pioglitazone, rosiglitazone). Non-limiting examples of cancer drugs that are known to inhibit angiogenesis include: Adriamycin, Cyclophosphamide, Docetaxel, Doxorubicin, Interferon alpha, Methotrexate, Paclitaxel, Thalidomide, Topotecan, and Vinblastine. Moreover, arthritis agents, such as Etanercept and Infliximab can inhibit angiogenesis.

Disclosed are methods for enhancing the angiogenic activity of a growth factor in a subject by administering to the subject a glypisome disclosed herein. Also disclosed are therapeutic angiogenesis methods that involve administering to a subject in need thereof a glypisome in combination with a growth factor. These methods can be used to treat any disease associated with insufficient blood supply. For example, the methods can be used to treat peripheral arterial disease (PAD), chronic wounds, or ischemic cardiovascular and cerebrovascular disorders (e.g., ischemic stroke). The methods can also be used for tissue regeneration, e.g., bone regeneration, or tissue/organ transplantation to promote vascularization.

Peripheral Arterial Disease (PAD) is a term that covers an array of medical problems caused by obstruction of the large arteries in the arms or legs. PVD can result from atherosclerosis, inflammatory processes leading to stenosis, an embolism, or thrombus formation. It causes either acute or chronic ischemia (lack of blood supply). A more severe form of PAD is critical limb ischemia (CLI), a leading cause of lower limb amputations. The Angiogenesis Foundation estimates that 1.4 million people in the United States have CLI, with an estimated 350,876 new cases diagnosed each year. Smoking, high cholesterol, and high blood pressure are also significant risk factors for PAD and CLI.

There are three main types of chronic wounds: venous ulcers, diabetic ulcers, and pressure ulcers. Venous ulcers usually occur in the legs, account for the majority of chronic wounds, and mostly affect the elderly. They are caused by improper function of tiny valves in the veins that normally prevent blood from flowing backward. The dysfunction of these valves impedes the normal circulation of blood in the legs, causing tissue damage and impaired wound healing. Diabetic patients are particularly susceptible to developing ulcers. People with advanced diabetes have a diminished perception of pain in the extremities due to nerve damage, and therefore may not initially notice small scratches or bruises on their legs and feet. Diabetes also impairs the immune system and damages capillaries. Repeated injury, compounded by impaired healing, can cause even the smallest cut or bruise to become dangerously infected. Pressure ulcers comprise the third main type of chronic wounds. These typically occur in people who are bedridden or whose mobility is severely limited. Pressure ulcers are caused by a loss of blood circulation that occurs when pressure on the tissue is greater than the pressure in capillaries, thereby cutting off circulation. Parts of the body that are particularly susceptible to pressure ulcers include the heels, shoulder blades, and sacrum (the triangular bone at the base of the spine forming the posterior of the pelvis).

Currently available approaches for treating patients with ischemic heart disease include medical therapy or coronary revascularization by percutaneous coronary angioplasty (PCA) or coronary artery bypass grafting (CABG). However, a significant number of these patients are not candidates for coronary revascularization procedures or achieve incomplete revascularization with these procedures. Consequently, many of these patients have persistent symptoms of myocardial ischemia despite intensive medical therapy. The discovery of candidate molecules able to stimulate myocardial angiogenesis has stirred a growing interest in using these molecules for therapeutic application.

Preliminary clinical experiences suggest that therapeutic angiogenesis may provide additional blood flow to incompletely revascularized areas. More recently, several studies suggest that implanted bone marrow cells may induce angiogenesis in ischemic myocardium.

In some embodiments, the glypican used in these methods is glypican-1, which is the prevalent member of the glypican family in endothelial cells and the vascular system.

Methods for Promoting Nerve Regeneration

Also disclosed are methods for enhancing the nerve regenerative activity of a growth factor in a subject by administering to the subject a glypisome disclosed herein. Also disclosed are methods for promoting nerve regeneration that involve administering to a subject in need thereof a glypisome in combination with a neurotrophic growth factor.

Neurotrophins are a family of growth factors that are known to promote nerve cell growth and survival. Examples of neurotrophins include Nerve Growth Factor (NGF), bFGF, brain-derived neurotrophic factor (BDNF), insulin-like growth factor (IGF), ciliary neurotrophic factor (CNTF), and neurotrophic factor-4/5 (NT-4/5).

In some embodiments, the glypican used in these methods is any one or more of glypican-1 to glypican-6 (GPC1 to GPC6). For example, glypican-3 can be used with these methods.

Pharmaceutical Compositions

The disclosed glypisome compositions, including microcapsules comprising glypisomes and growth factors, can be used therapeutically in combination with a pharmaceutically acceptable carrier. Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art.

Pharmaceutical compositions can include carriers, thickeners, diluents, buffers, preservatives, surface active agents and the like in addition to the molecule of choice. Pharmaceutical compositions can also include one or more active ingredients such as antimicrobial agents, anti-inflammatory agents, anesthetics, and the like.

The pharmaceutical composition can be administered in a number of ways depending on whether local or systemic treatment is desired, and on the area to be treated. Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives can also be present such as, for example, antimicrobials, anti-oxidants, chelating agents, and inert gases and the like.

Some of the compositions can be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

The dosage ranges for the administration of the compositions are those large enough to produce the desired effect in which the symptoms disorder are affected. The dosage should not be so large as to cause adverse side effects, such as unwanted cross-reactions, anaphylactic reactions, and the like. Generally, the dosage will vary with the age, condition, sex and extent of the disease in the patient and can be determined by one of skill in the art. The dosage can be adjusted by the individual physician in the event of any counterindications. Dosage can vary, and can be administered in one or more dose administrations daily, for one or several days.

Administration

The disclosed glypisome can be administered in combination with one or more growth factors. For example, the method can involve administering to the subject a microcapsule comprising therapeutically effective amounts of a glypisome and a growth factor. The method can also involve sequential administration of the glypisome and growth factor, in either order.

The herein disclosed compositions, including pharmaceutical composition, may be administered in a number of ways depending on whether local or systemic treatment is desired, and on the area to be treated. For example, the disclosed compositions can be administered intravenously, intraperitoneally, intramuscularly, subcutaneously, intracavity, or transdermally. The compositions may be administered orally, parenterally (e.g., intravenously), by intramuscular injection, by intraperitoneal injection, transdermally, extracorporeally, ophthalmically, vaginally, rectally, intranasally, topically or the like, including topical intranasal administration or administration by inhalant.

### Definitions

The term “alginate” refers to linear polysaccharides formed from β-D-mannuronate and β-L-guluronate in any M/G ratio, as well as salts and derivatives thereof.

The term “amino acid sequence” refers to a list of abbreviations, letters, characters or words representing amino acid residues. The amino acid abbreviations used herein are conventional one letter codes for the amino acids and are expressed as follows: A, alanine; B, asparagine or aspartic acid; C, cysteine; D aspartic acid; E, glutamate, glutamic acid; F, phenylalanine; G, glycine; H histidine; I isoleucine; K, lysine; L, leucine; M, methionine; N, asparagine; P, proline; Q, glutamine; R, arginine; S, serine; T, threonine; V, valine; W, tryptophan; Y, tyrosine; Z, glutamine or glutamic acid.

The term “biocompatible” refers to a material and any metabolites or degradation products thereof that are generally non-toxic to the recipient and do not cause any significant adverse effects to the subject.

The term “carrier” means a compound, composition, substance, or structure that, when in combination with a compound or composition, aids or facilitates preparation, storage, administration, delivery, effectiveness, selectivity, or any other feature of the compound or composition for its intended use or purpose. For example, a carrier can be selected to minimize any degradation of the active ingredient and to minimize any adverse side effects in the subject.

The term “glypisome” refers to a protovesicle comprising a glypican protein.

The term “hydrogel” refers to a substance formed when an organic polymer (natural or synthetic) is cross-linked via covalent, ionic, or hydrogen bonds to create a three-dimensional open-lattice structure which entraps water molecules to form a gel. Biocompatible hydrogel refers to a polymer that forms a gel which is not toxic to living cells, and allows sufficient diffusion of oxygen and nutrients to the entrapped cells to maintain viability.

The term “lipid vesicle” refers to a small vesicle composed of various types of lipids, phospholipids and/or surfactant which can be embedded with a glypisome disclosed herein.

The term “liposome” refers to vesicle composed of a lipid bilayer.

The term “micelle” refers to vesicle composed of a lipid monolayer.

The term “microcapsule” refers to a particle or capsule having a mean diameter of about 50 μm to about 1000 μm, formed of a cross-linked hydrogel shell surrounding a biocompatible matrix. The microcapsule may have any shape suitable for cell encapsulation. The microcapsule may contain one or more cells dispersed in the biocompatible matrix, cross-linked hydrogel, or combination thereof, thereby “encapsulating” the cells.

The terms “peptide,” “protein,” and “polypeptide” are used interchangeably to refer to a natural or synthetic molecule comprising two or more amino acids linked by the carboxyl group of one amino acid to the alpha amino group of another. In addition, the terms refer to amino acids joined to each other by peptide bonds or modified peptide bonds, e.g., peptide isosteres, etc. and may contain modified amino acids other than the 20 gene-encoded amino acids. The polypeptides can be modified by either natural processes, such as post-translational processing, or by chemical modification techniques which are well known in the art. Modifications can occur anywhere in the polypeptide, including the peptide backbone, the amino acid side-chains and the amino or carboxyl termini. The same type of modification can be present in the same or varying degrees at several sites in a given polypeptide. Also, a given polypeptide can have many types of modifications. Modifications include, without limitation, acetylation, acylation, ADP-ribosylation, amidation, covalent cross-linking or cyclization, covalent attachment of flavin, covalent attachment of a heme moiety, covalent attachment of a nucleotide or nucleotide derivative, covalent attachment of a lipid or lipid derivative, covalent attachment of a phosphytidylinositol, disulfide bond formation, demethylation, formation of cysteine or pyroglutamate, formylation, gamma-carboxylation, glycosylation, GPI anchor formation, hydroxylation, iodination, methylation, myristolyation, oxidation, pergylation, proteolytic processing, phosphorylation, prenylation, racemization, selenoylation, sulfation, and transfer-RNA mediated addition of amino acids to protein such as arginylation.

The term “percent (%) sequence identity” or “homology” is defined as the percentage of nucleotides or amino acids in a candidate sequence that are identical with the nucleotides or amino acids in a reference nucleic acid sequence, after aligning the sequences and introducing gaps, if necessary, to achieve the maximum percent sequence identity. Alignment for purposes of determining percent sequence identity can be achieved in various ways that are within the skill in the art, for instance, using publicly available computer software such as BLAST, BLAST-2, ALIGN, ALIGN-2 or Megalign (DNASTAR) software. Appropriate parameters for measuring alignment, including any algorithms needed to achieve maximal alignment over the full-length of the sequences being compared can be determined by known methods.

The term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problems or complications commensurate with a reasonable benefit/risk ratio.

The term “promote” refers to an increase in an activity, response, condition, disease, or other biological parameter. This can include but is not limited to the initiation of the activity, response, condition, or disease. This may also include, for example, a 10% increase in the activity, response, condition, or disease as compared to the native or control level. Thus, the reduction can be a 10, 20, 30, 40, 50, 60, 70, 80, 90, 100%, or any amount of increase in between as compared to native or control levels.

The term “proteovesicle” refers to lipid vesicle comprising a protein embedded or attached to its surface or the vesicle/micelle formed through the self association of glypican proteins with or without native lipids from the cell membrane.

The term “operably linked to” refers to the functional relationship of a nucleic acid with another nucleic acid sequence. Promoters, enhancers, transcriptional and translational stop sites, and other signal sequences are examples of nucleic acid sequences operably linked to other sequences. For example, operable linkage of DNA to a transcriptional control element refers to the physical and functional relationship between the DNA and promoter such that the transcription of such DNA is initiated from the promoter by an RNA polymerase that specifically recognizes, binds to and transcribes the DNA.

The term “subject” refers to any individual who is the target of administration or treatment. The subject can be a vertebrate, for example, a mammal. Thus, the subject can be a human or veterinary patient. The term “patient” refers to a subject under the treatment of a clinician, e.g., physician.

The term “therapeutically effective” refers to the amount of the composition used is of sufficient quantity to ameliorate one or more causes or symptoms of a disease or disorder. Such amelioration only requires a reduction or alteration, not necessarily elimination.

The terms “transformation” and “transfection” mean the introduction of a nucleic acid, e.g., an expression vector, into a recipient cell including introduction of a nucleic acid to the chromosomal DNA of said cell.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

The term “variant” refers to an amino acid or peptide sequence having conservative amino acid substitutions, non-conservative amino acid subsitutions (i.e. a degenerate variant), substitutions within the wobble position of each codon (i.e. DNA and RNA) encoding an amino acid, amino acids added to the C-terminus of a peptide, or a peptide having 60%, 70%, 80%, 90%, 95%, 96%, 97%, 98%, or 99% sequence identity to a reference sequence.

The term “vector” or “construct” refers to a nucleic acid sequence capable of transporting into a cell another nucleic acid to which the vector sequence has been linked. The term “expression vector” includes any vector containing a gene construct in a form suitable for expression by a cell (e.g., operably linked to a transcriptional control element).

A number of embodiments of the invention have been described. Nevertheless, it will be understood that various modifications may be made without departing from the spirit and scope of the invention. Accordingly, other embodiments are within the scope of the following claims.

### EXAMPLES

### Example 1: Glypisomes: A Construct for Enhancing of Growth Factor Activity for Therapeutic Angiogenesis

Methods

Cell Culture: Human umbilical vein endothelial cells (HUVECs) were cultured under standard culture conditions and were used to analyze the angiogenic effects on endothelial cells in the in vitro assays. HUVECs that were used for these experiments did not exceed passage 6.

Cell proliferation assay: 2500 cells per well were plated in a 96 well plate. HUVEC's were then serum starved for 24 hours. The serum starve media is the same as the regular media except it only has 2% FBS and does not contain the added growth factors or heparin. Glypican proteoliposomes and growth factors were then added to each well. Proliferation was assessed 36 hours later using the Cell Signaling BrdU kit.

Wound healing cell migration assay: HUVEC cells were cultured until confluent in 6 well plates. The cells were then serum starved for 24 hours. A cell scraper was used to create a cross shaped wound in each well. The glypican-proteoliposomes and FGF (10 ng/ml) were added to the media immediately after the wounds were made. The wounds were imaged at 0 and 16 hours. The average distance of the wounds was calculated using Metamorph software (Molecular Devices). The distance traveled was calculated by taking the difference of these two measurements.

Tubule formation assay: 24 well plates were coated with matrigel and allowed to gel for 1 hour at 37° C. HUVECs were plated at 2×10⁴ cells per well in the 24 well plates. They were then incubated for 16 hours at 37° C. and imaged. Average tubule length, number of tubules, and number of tubule branching points was then quantified using Metamorph software (Molecular Devices).

Production and isolation of recombinant glypican-1: HeLa cells were transfected to express recombinant his-tagged GPC-1 and selected by resistance to puromycin. GPC-1 was then isolated with a his-tag spin column (GE) to a final concentration of 61 μg/ml. Purity was confirmed by silver stain and western blot.

Synthesis of glypican-1 proteoliposomes Lipid stock solutions were dissolved at 10 mg/ml in chloroform. 1,2-dioleoyl-sn-glycero-3-phosphocholine (DOPC), 1,2-dioleoyl-sn-glycero-3-phosphoethanolamine (DOPE), cholesterol and sphingomyelin were mixed together at a 40:20:20:20 ratio, respectively, in a round bottom flask. The solvent was removed by rotovap to and the lipid film was dried with argon gas. The film was resuspended in HEPES buffer by vortexing, sonicating and freeze thawing to achieve a 12.3 mM final lipid solution. The lipid solution was then extruded through a 400 nm polycarbonate membrane. Varying volumes of the lipid solution was added to varying volumes of glypican (61.1 μg/ml). 1% n-octyl-β-D-glucopyranoside was added to the lipid and protein solution. Every 30 minutes the concentration of the solution was reduced 10% by adding PBS to a final concentration of 40%. The excess protein and detergent were then removed by dialysis in PBS at 4° C. overnight with Biobeads.

Alginate bead encapsulation of glypican-1 proteoliposomes: 4% sodium alginate solution and 0.85% NaCl were mixed together at equal volumes. The proteoliposomes (1:100 dilution) and FGF (0.75 μg/100 μl) were added. The alginate solution was then extruded through a 30-gauge needle into a 1.1% CaCl solution and allowed to crosslink for 1 hour at 4 C.

Animal model of hindlimb ischemia: C57-B6 mice were anesthetized with 2% isofluorane gas. The femoral artery was separated from the femoral vein nerve, then tied off with silk sutures and ligated at two points 1 cm apart. 24 alginate beads (either FGF or FGF and glypican proteoliposomes) were then implanted along the femoral artery in the incision. The incision was closed with vicryl sutures. Relative blood flow was measured at days 1, 3, 5, 7, and 14 using laser speckle imaging. The mice were sacrificed on day 14. The hindlimb muscles were harvested and frozen in isopentane in a liquid nitrogen bath. These tissue samples were stored at −80° C. until they were fixed at a later date.

Laser speckle imaging: The mice were anesthetized and their hind paws were illuminated with 785 nm diode laser (Thorlabs). Speckle contrast images were taken and converted in MATLAB. The relative intensity of the speckle contrast images was measured in using metamorph.

Statistical Analysis. All results are shown as mean±standard error of the mean. Comparisons between only two groups were performed using a 2-tailed Student's t-test. Differences were considered significant at p<0.05. Multiple comparisons between groups were analyzed by 2-way ANOVA followed by a Dunnett post-hoc test comparing to the control and growth factor alone treatment groups. A 2-tailed probability value <0.05 was considered statistically significant.

Results

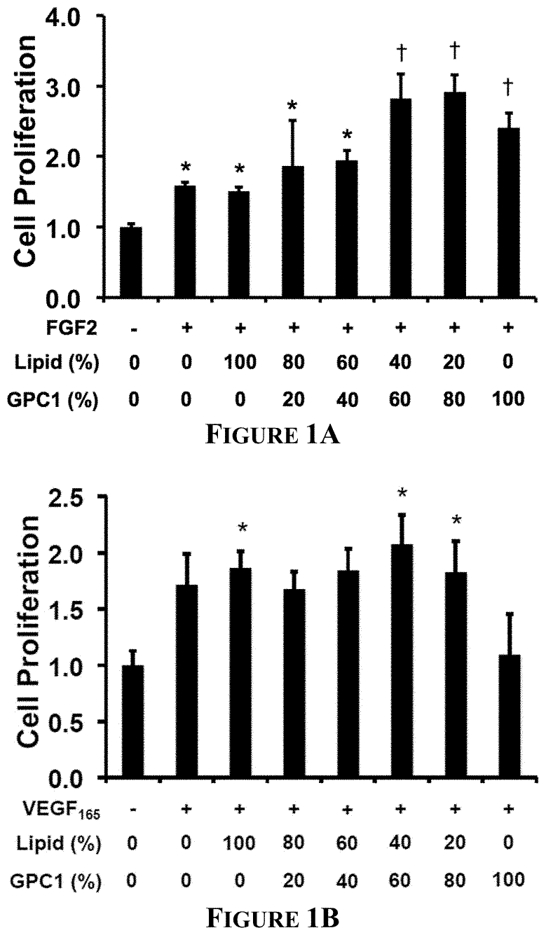

Glypican-1 Proteoliposomes enhance FGF-2 but not VEGF-induced proliferation in endothelial cells. Recombinant glypican-1 was isolated and embedded in a liposomes using progressive detergent extraction. It was first examined whether the exogenous delivery of glypican-1 in a proteoliposome format (glypisomes) were capable of enhancing FGF-2 and VEGF-induced proliferation in cultured endothelial cells. To optimize the composition of the glyposomes, they were made with varying ratios between the glypican-1 protein and the lipid. To examine the extent to which glypican incorporation modified the liposomes, the size and charge of the glypisomes were measured using dynamic light scattering (DLS) (FIG. 9). As an initial screen for effectiveness, the ability of these carrying composition glypisomes to enhance FGF-2 and VEGF activity was measured in a proliferation assay. For FGF-2, mid-range composition glypisomes increased proliferation to the same level of FGF by nearly three-fold (FIG. 1A). Interestingly, free glypican and the liposomes alone each increased proliferation but not to the extent as the mid range glypisomes. In contrast VEGF induced proliferation was not enhanced by any of the liposomes (FIG. 1B). When delivered in combination with FGF-2, glypican-1 proteoliposomes increased endothelial cell proliferation by nearly three-fold. For VEGF, the glypican-1 proteoliposomes had no significant effects on endothelial cell proliferation.

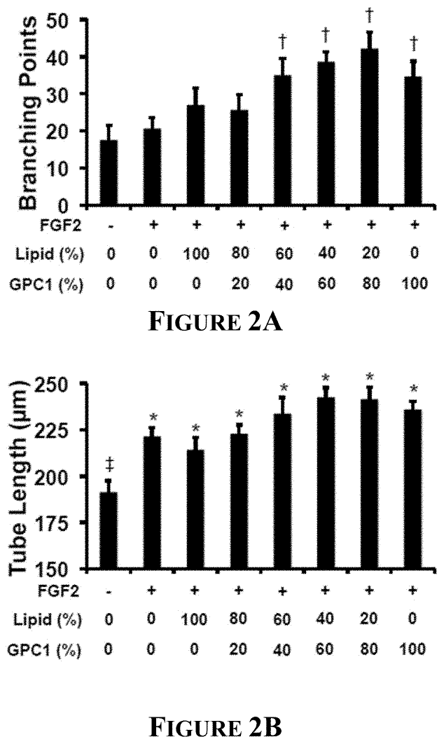

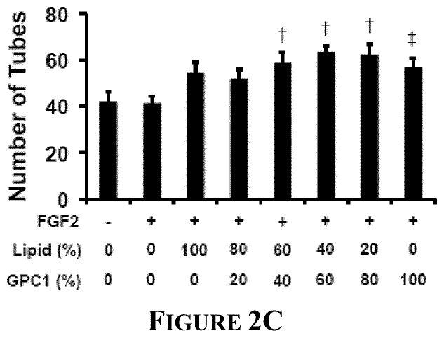

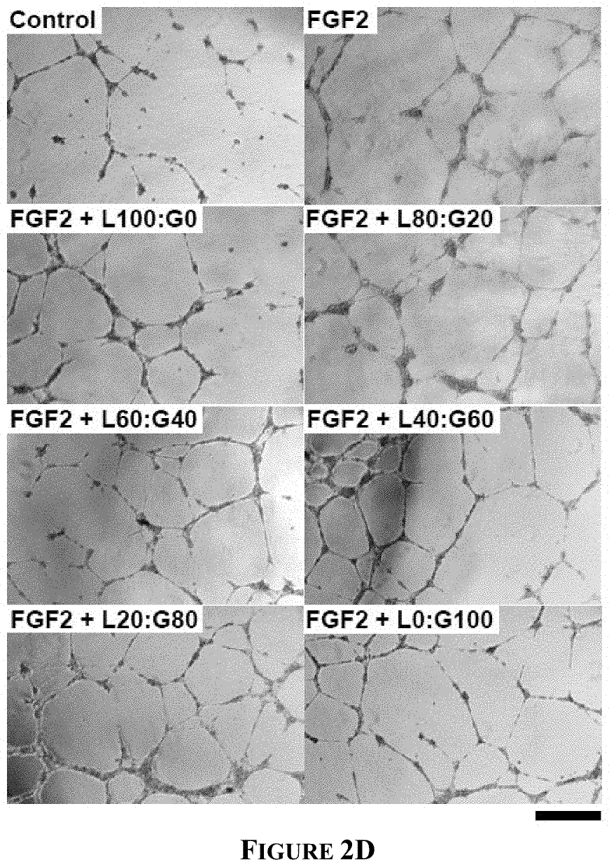

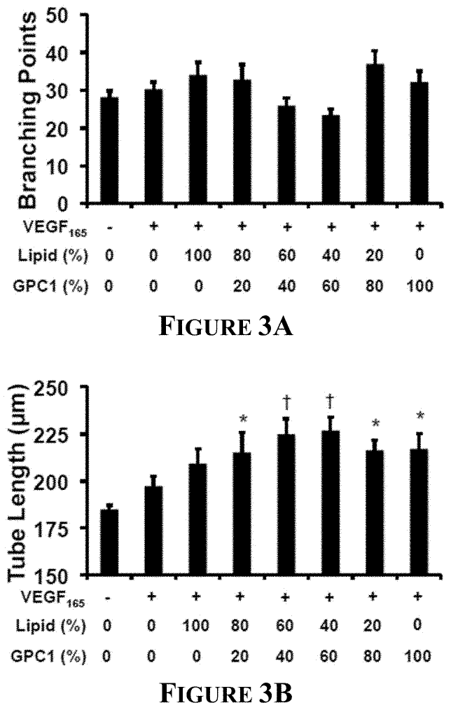

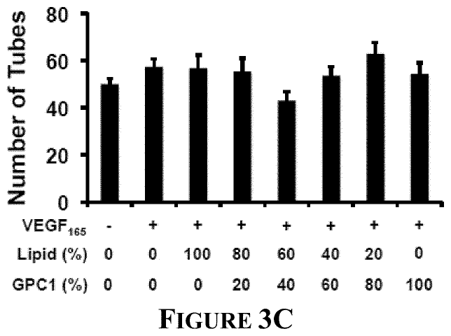

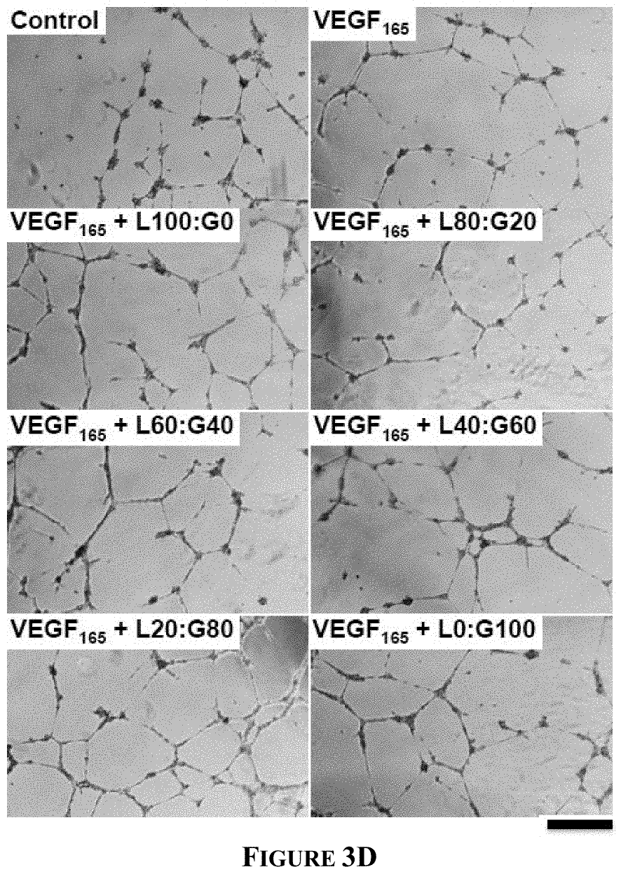

Glypican-1 Proteoliposomes enhance FGF-2 branch point formation in an in-vitro tube formation assay. The effects of glypisome treatment in enhancing growth factor activity was next examined using an in vitro tube formation assay. In this assay, endothelial cells grown on matrigel were starved and then treated with FGF-2 or FGF-2 in combination with the glypisomes of varying protein to lipid ratio. Included in analysis was the liposomes alone (L100:G0) and isolated glypican-1 alone (L0:G100). For the four highest glypican-1 containing glypisomes there was increased tube formation including increased branch points (FIG. 2A), tube length (FIG. 2B), and tube number (FIG. 2C) in the tube network forms. Interestingly, when glypisomes were delivered with VEGF₁₆₅ there was only enhanced tube length in the midrange (60:40 and 40:60 lipid to protein ratio) glypisomes. There were no significant alterations in the number of branch points and number of tubes with glypisome treatment in combination with VEGF.

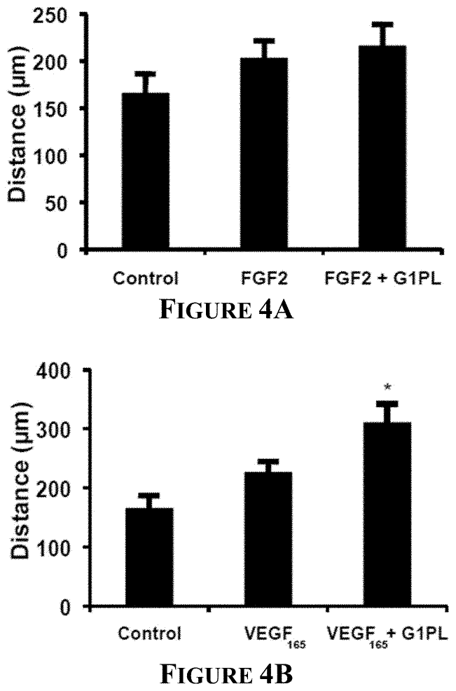

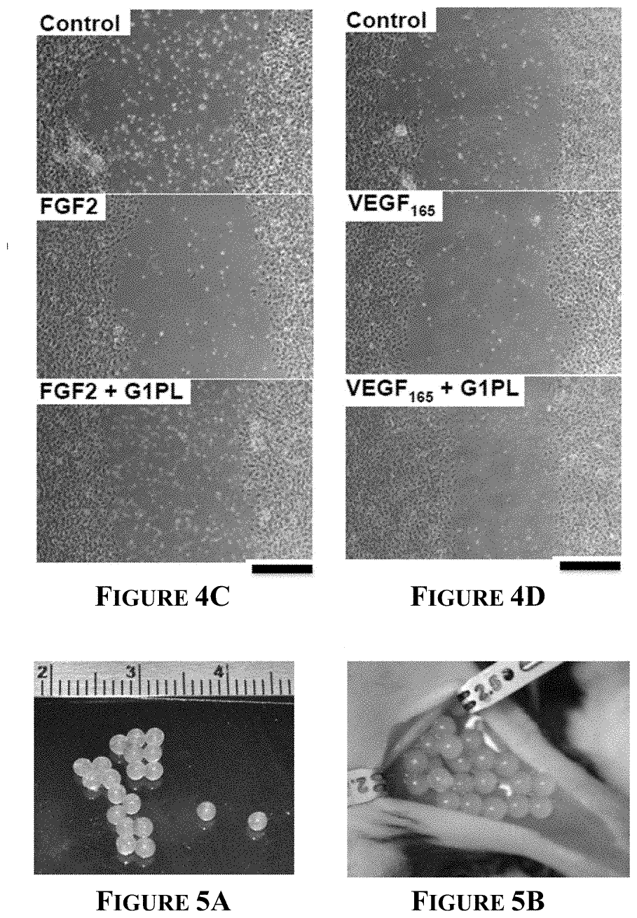

Glypican-1 Proteoliposomes enhance VEGF induced migration. It was next examined whether the optimal concentration glypisomes were also capable of increasing the mitogenic properties of FGF-2 and VEGF. Scratch wounds were created in monolayers of post-confluent endothelial cells in the presence of growth factors alone or in combination with the optimal composition glypisomes. Enhancement of FGF-2 migration was not observed, but there was an increase in the wound edge migration rate with glypisomes together with VEGF (FIGS. 4A to 4D).

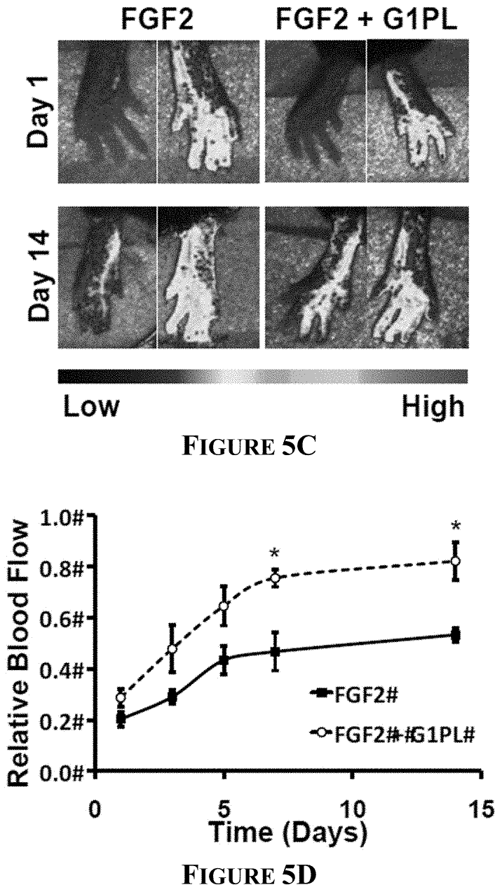

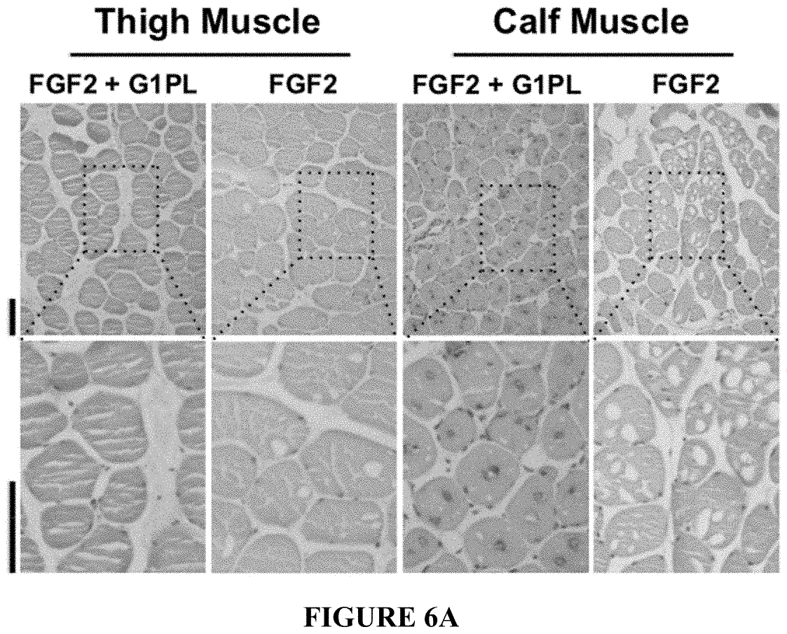

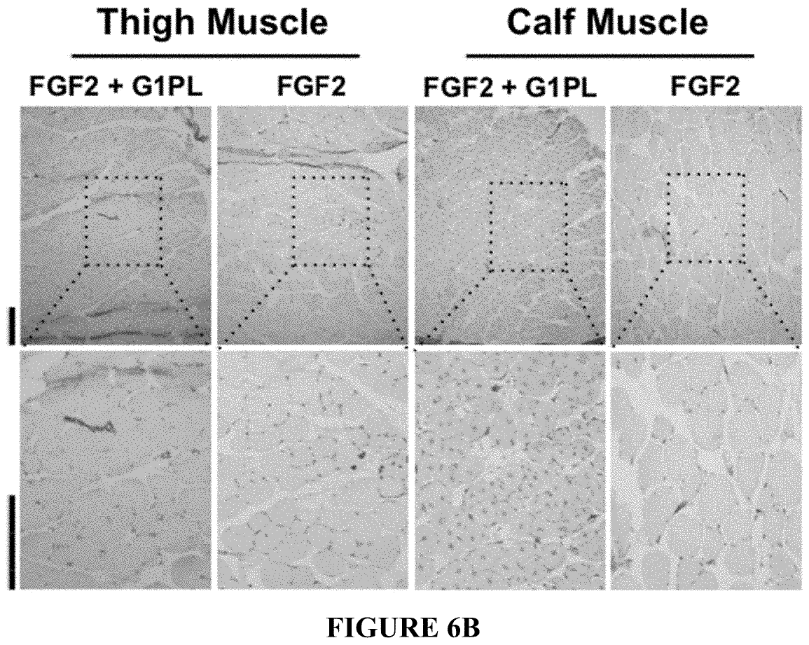

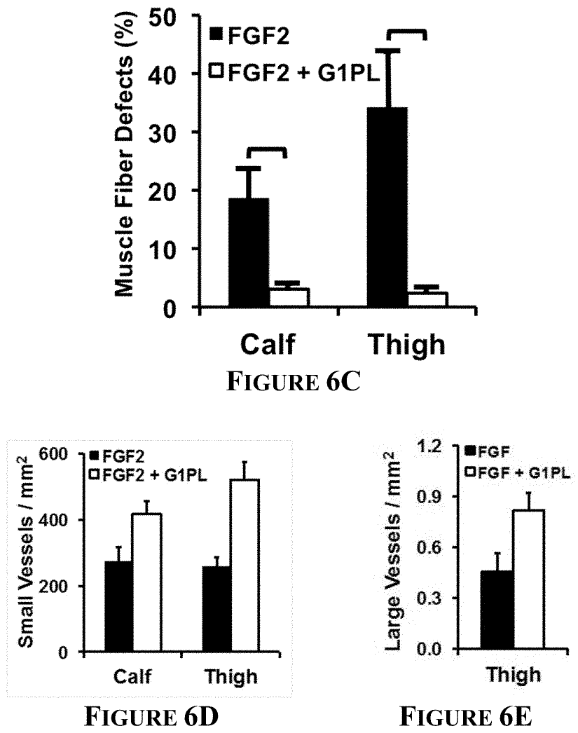

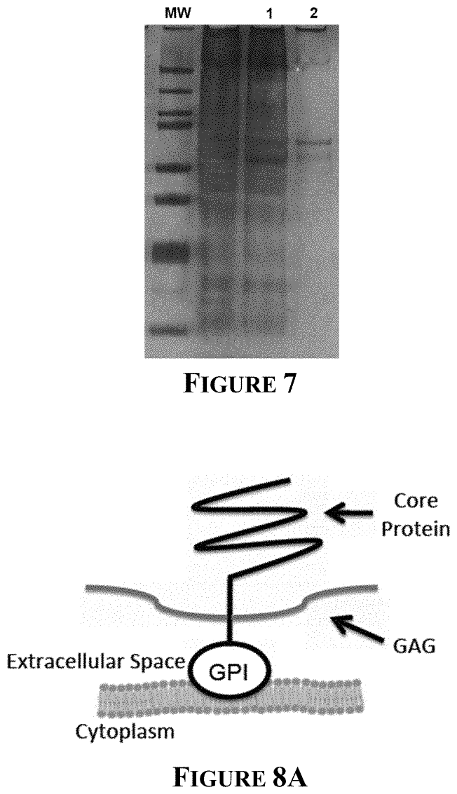

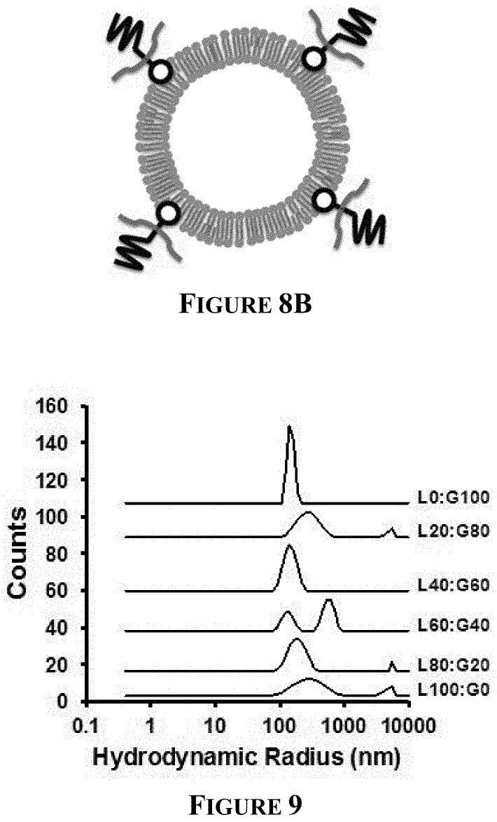

Alginate encapsulated glypican-1 proteoliposomes enhance revascularization of the mouse ischemic hind limb. Both exogenous FGF-2 and VEGF have been shown to enhance revascularization in animal models of ischemia (Laham, R. J., et al. J Pharmacol Exp Ther (2000) 292:795-802; Baffour, R., et al. J Vasc Surg (1992) 16:181-191). Hind limb ischemia was induced in mice by ligating the femoral artery. FGF-2 and FGF-2 in combination with the optimized glypisomes was delivered by encapsulating them in an alginate carrier. Perfusion in the ischemic hind limb and contralateral control limb was monitored for the recovery of perfusion for 14 days. The glypisomes enhanced FGF-2 activity leading to nearly twice the relative perfusion in the ischemic limb of glypisome treated mice (FIG. 5D). Histological analysis of the calf and thigh muscles from the mice demonstrated reduced formation of ischemic changes including loss of muscle fibers (FIGS. 6A and 6B). Immunohistochemical staining for PECAM-1 and subsequent analysis demonstrated increased capillaries in the thigh and calf muscles. An analysis of larger vessels revealed increased arteriogenesis in the thigh muscle of glypican treated mice. Together these results support that glypisomes can enhance FGF-2 therapy in-vivo.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

1.-15. (canceled)

16. A method for enhancing the angiogenic or wound healing activity of a growth factor in a subject, comprising administering to a subject in need thereof a composition comprising, a biodegradable microcapsule or microbead encapsulating therein effective amounts of a growth factor and a proteovesicle comprising a recombinant glypican polypeptide embedded in a lipid vesicle; wherein the microcapsule or microbead comprises a biocompatible hydrogel; and wherein the lipid vesicle is prepared through the self-association of the glypican polypeptide.

17. The method of claim 16, wherein the subject has been diagnosed with peripheral arterial disease (PAD), a chronic wound, or an ischemic cardiovascular or cerebrovascular disorder.

18. A method for promoting angiogenesis in a subject, comprising administering to a subject in need thereof a composition comprising, a biodegradable microcapsule or microbead encapsulating therein effective amounts of a growth factor and a proteovesicle comprising a recombinant glypican polypeptide embedded in a lipid vesicle; wherein the microcapsule or microbead comprises a biocompatible hydrogel; and wherein the lipid vesicle is prepared through the self-association of the glypican polypeptide.

19. The method of claim 18, wherein the subject has been diagnosed with peripheral arterial disease (PAD), a chronic wound, or an ischemic cardiovascular or cerebrovascular disorder.

20. A method for promoting nerve regeneration in a subject, comprising administering to a subject in need thereof a therapeutically effective amount of a composition comprising, a biodegradable microcapsule or microbead encapsulating therein effective amounts of a growth factor and a proteovesicle comprising a recombinant glypican polypeptide embedded in a lipid vesicle; wherein the microcapsule or microbead comprises a biocompatible hydrogel; and wherein the lipid vesicle is prepared through the self-association of the glypican polypeptide.

21. The method of claim 16, wherein the biocompatible hydrogel comprises a polysaccharide.

22. The method of claim 16, wherein the biocompatible hydrogel comprises alginate.

23. The method of claim 16, wherein the microcapsule or microbead is from 1 μm in diameter up to 3 mm in diameter.

24. The method of claim 16, wherein the growth factor is a heparin-binding growth factor.

25. The method of claim 16, wherein the growth factor is an angiogenesis-related or wound healing-related growth factor.

26. The method of claim 25, wherein the angiogenesis-related growth factor comprises a fibroblast growth factor (FGF), platelet-derived growth factor (PDGF), vascular endothelial growth factor (VEGF), and Placental growth factor (PlGF).

27. The method of claim 24, wherein the heparin-binding growth factor is a neurotrophic heparin-binding growth factor.

28. The method of claim 27, wherein the neurotrophic heparin-binding growth factor is selected from the group consisting of a nerve growth factor (NGF), FGF, brain-derived neurotrophic factor (BDNF), insulin-like growth factor (IGF), ciliary neurotrophic factor (CNTF), and neurotrophic factor-4/5 (NT-4/5).

29. The method of claim 18, wherein the biocompatible hydrogel comprises a polysaccharide.

30. The method of claim 18, wherein the biocompatible hydrogel comprises alginate.

31. The method of claim 18, wherein the growth factor is an angiogenesis-related or wound healing-related growth factor.

32. The method of claim 31, wherein the angiogenesis-related growth factor comprises a fibroblast growth factor (FGF), platelet-derived growth factor (PDGF), vascular endothelial growth factor (VEGF), and Placental growth factor (PlGF).

33. The method of claim 20, wherein the biocompatible hydrogel comprises a polysaccharide.

34. The method of claim 20, wherein the biocompatible hydrogel comprises alginate.
